(19) United States
(12) Patent Application Publication
(10) Pub. No.: US 2023/0174657 A1
(43) Pub. Date: Jun. 8, 2023

(54) COMPOSITIONS COMPRISING AN ANTIBODY AGAINST INTERLEUKIN-6 RECEPTOR FOR THE TREATMENT OF RHEUMATOID ARTHRITIS AND METHODS OF USING SAME

(71) Applicant: SANOFI BIOTECHNOLOGY, Paris (FR)

(72) Inventors: Inka ALBRECHT, Frankfurt am Main (DE); Eugen FEIST, Frankfurt am Main (DE); Peer-Malte ARIES, Frankfurt am Main (DE); Silke ZINE, Frankfurt am Main (DE); Harald BURKHARDT, Frankfurt am Main (DE); Hans-Peter TONEY, Frankfurt am Main (DE)

(21) Appl. No.: 17/926,018

(22) PCT Filed: May 25, 2021
(86) PCT No.: PCT/IB2021/054546

§ 371 Date: Nov. 17, 2022

Related U.S. Application Data
(60) Provisional application No. 63/155,125, filed on Mar. 1, 2021.
(60) Provisional application No. 63/030,065, filed on May 26, 2020.

(30) Foreign Application Priority Data
May 25, 2021 (EP) 21315087.3

(51) Int. Cl. C07K 16/28; A61P 19/02; A61K 39/395; A61K 31/519; A61P 29/00

(52) U.S. Cl. CPC C07K 16/2866; A61P 19/02; A61K 39/3955; A61K 31/519; A61P 29/00; A61K 2039/505

(57) ABSTRACT

The present technology provides compositions and methods of treating and improving the symptoms of rheumatoid arthritis using an antibody or antigen-binding fragment thereof that specifically binds human interleukin-6 receptor (hIL-6R).

### RELATED APPLICATIONS

This application claims priority to U.S. Provisional Patent Application Serial Nos. 63/030,065, filed May 26, 2020, and 63/155,125, filed Mar. 1, 2021, and EP Provisional Patent Application Serial No. 21315087.3, filed May 25, 2021. The entire disclosure of each of these applications is hereby incorporated herein by reference in its entirety.

### FIELD

The present technology relates to the field of therapeutic treatment of rheumatoid arthritis. More specifically, the present technology relates to the use of interleukin-6 receptor (IL-6R) antagonists, such as anti-IL-6R antibodies, to treat rheumatoid arthritis.

### BACKGROUND

It is estimated that approximately 0.5% to 1% of the adult population in North America and Europe is affected by rheumatoid arthritis (RA). RA affects women twice as often as men and the incidence is highest among women over 40 years of age.

RA is characterized by persistent synovitis and progressive destruction of cartilage and bone in multiple joints. The hallmark of the disease is a symmetric polyarthritis characteristically involving the small joints of the hands and feet. The inflammatory process can also target other organs, characteristically bone marrow (anemia), eye (scleritis, episcleritis), lung (interstitial pneumonitis, pleuritis), cardiac (pericarditis) and skin (nodules, leukocytoclastic vasculitis). Systemic inflammation is characterized by laboratory abnormalities, such as anemia, elevated erythrocyte sedimentation rate, fibrinogen and C-reactive protein (CRP) and by clinical symptoms of fatigue, weight loss, muscle atrophy in affected joint areas. The presence of polyclonal high-titer rheumatoid factors and anticyclic citrullinated peptide (anti-CCP) antibodies provides evidence of immune dysregulation. It has been estimated that 65% to 70% of RA subjects have progressive disease that leads to joint destruction, disability and premature death.

In addition to improving the clinical symptoms of RA subjects, there is a growing interest in improving the physical function and the health-related quality of life of RA subjects. Indeed, in addition to the usual instruments intended to assess health status of the subjects (presence or absence of disease), physicians and clinicians developed new instruments to measure the physical function and quality of life of the subjects, which are useful parameters for the physician to assess the overall response of his or her subject to a specific treatment. Quality of life, for instance, goes beyond the impairment/disability and handicap continuum by asking what subjects' health status prevents them from doing and also about their emotional response to these restrictions. Quality of life also reflects the influences of the personal, social and economic resources that an individual has and the way in which these interact with health status (British Journal of Rheumatology 1997; 36:884-888). The physical function assessment of RA subjects typically takes into account the fine movements of the upper extremity, locomotor activities of the lower extremity, and activities that involve both the upper and lower extremities. These parameters are now widely used by the physicians, clinicians and regulatory agencies to compare the different treatment options offered to RA subjects. In certain instances, two treatments with a similar efficacy profile may have different quality of life or physical function improvement profiles.

There is a need in the art for improved treatment regimens for the improvement of symptoms associated with RA.

### SUMMARY

The present disclosure provides methods of treating rheumatoid arthritis in a subject in need thereof. The methods include administering to the subject an effective amount of sarilumab (SAR153191). In certain embodiments, the subject was previously ineffectively treated for rheumatoid arthritis by administering a Janus kinase (JAK)-inhibitor (JAKi).

In one aspect, the disclosure provides methods of treating rheumatoid arthritis in a subject that was previously ineffectively treated for rheumatoid arthritis with a JAKi comprising administering an antibody that specifically binds to a human IL-6 receptor and comprises a heavy chain variable region (VH) and a light chain variable region (VL), wherein the VH comprises the three complementarity determining regions (CDRs) found within the amino acid sequence of SEQ ID NO:2 and wherein the VL comprises the three CDRs found within the amino acid sequence of SEQ ID NO:3.

In one embodiment, the subject is also administered a therapeutically effective amount of methotrexate. In one embodiment, the antibody is administered subcutaneously at a dose from about 150 mg to about 200 mg once every two weeks. In one embodiment, methotrexate is administered at a dose from 6 mg to 25 mg every week. In another embodiment, the antibody comprises three heavy chain CDR (HCDR) sequences comprising SEQ ID NOs: 4, 5, and 6 and three light chain CDR (LCDR) sequences comprising SEQ ID NOs: 7, 8 and 9. In a specific embodiment, the antibody is sarilumab. In one embodiment, the subject achieves a DAS28-ESR score below 3.1 after at least 12 weeks of administration of the antibody. In one embodiment, the subject achieves a clinical disease activity index (CDAI) score below 16 after at least 12 weeks of administration of the antibody. In some embodiments, the subject was previously ineffectively treated for rheumatoid arthritis with a JAKi selected from the group consisting of baricitinib, tofacitinib, and upadacitinib.

In another aspect, the present disclosure provides methods of treating rheumatoid arthritis in a subject that was previously ineffectively treated for rheumatoid arthritis with a JAKi, comprising administering a disease modifying antirheumatic drug (DMARD) selected from the group consisting of methotrexate, sulfasalazine, hydroxychloroquine and leflunomide, and an antibody that specifically binds to a human IL-6 receptor and comprises a VH and a VL, wherein the VH comprises the three CDRs found within the amino acid sequence of SEQ ID NO:1 and wherein the VL comprises the three CDRs found within the amino acid sequence of SEQ ID NO:2.

In one embodiment, the antibody comprises three HCDR sequences comprising SEQ ID NOs: 4, 5, and 6 and 5, and three LCDR sequences comprising SEQ ID NOs: 7, 8 and 9. In one embodiment, the DMARD that is administered is methotrexate. In one embodiment, the antibody is administered subcutaneously at a dose from about 150 mg to about 200 mg once every two weeks. In one embodiment, methotrexate is administered at a dose from 6 mg to 25 mg every week. In a specific embodiment, the antibody is sarilumab. In one embodiment, the subject achieves a DAS28-ESR score below 3.6 after at least 12 weeks of administration of the antibody and DMARD. In one embodiment, the subject achieves a CDAI score below 16 after at least 12 weeks of administration of the antibody and DMARD. In some embodiments, the subject was previously ineffectively treated for rheumatoid arthritis with a JAKi selected from the group consisting of baricitinib, tofacitinib, and upadacitinib.

Other embodiments will become apparent from a review of the ensuing detailed description, drawings, tables and accompanying claims.

### BRIEF DESCRIPTION OF THE DRAWINGS

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 graphically depicts DAS-28ESR, CDAI, TJC and SJC in subjects at baseline, and at 4-8 weeks or 12 weeks of JAKi treatment or control.

FIG. 2 depicts comorbidities and systemic manifestations at start of documentation.

FIG. 3 depicts an overview of the present study.

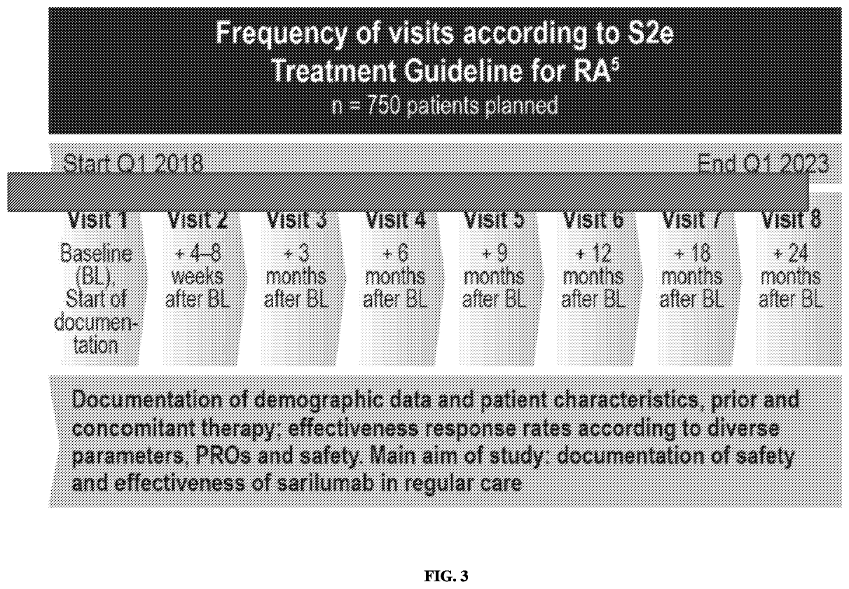

FIG. 4A-FIG. 4B depict the efficacy of sarilumab according to CDAI (A) and HAQ-DI (B) in patients with JAKi (JAKi-IR) or Treatment A (TA) as last prior treatment to sarilumab, in patients who have received either TNFi OR non-TNFi any time in treatment history (bDMARD TH), and in patients who have never received bDMARDs or tsDMARDs (b/tsDMARD naive). Full analysis population is shown. Therefore, baseline (BL) values might differ from baseline values in Table 8.

### DETAILED DESCRIPTION

The disclosure provides pharmaceutical compositions and methods of using the compositions for the treatment of rheumatoid arthritis (RA) in patients who have been previously treated with one or more JAKis. In some embodiments, patients were previously ineffectively treated with one or more JAKis. In some embodiments, the disclosed compositions and methods are for the improvement of at least one symptom of RA. The disclosed compositions include at least one antibody that specifically binds human interleukin-6 receptor (hIL-6R). In some embodiments, the disclosed compositions are effective for improving the physical function and the Quality of Life of subjects suffering from rheumatoid arthritis.

Anti-hIL-6R Antibodies

The present disclosure includes methods that comprise administering to a subject an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the subject binds specifically to the extracellular domain of hIL-6R. The extracellular domain of hIL-6R is shown in the amino acid sequence of SEQ ID NO: 1.

The amino acid sequence of SEQ ID NO: 1 is:

MVAVGCALLAALLAAPGAALAPRRCPAQEVARGVLTSLPGDSVTLTCPGV
EPEDNATVHWVLRKPAAGSHPSRWAGMGRRLLLRSVQLHDSGNYSCYRAG
RPAGTVHLLVDVPPEEPQLSCFRKSPLSNVVCEWGPRSTPSLTTKAVLLV
RKFQNSPAEDFQEPCQYSQESQKFSCQLAVPEGDSSFYIVSMCVASSVGS
KFSKTQTFQGCGILQPDPPANITVTAVARNPRWLSVTWQDPHSWNSSFYR
LRFELRYRAERSKTFTTWMVKDLQHHCVIHDAWSGLRHVVQLRAQEEFGQ
GEWSEWSPEAMGTPWTESRSPPAENEVSTPMQALTTNKDDDNILFRDSAN
ATSLPVQD.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Each heavy chain comprises a heavy chain variable region (abbreviated herein as HCVR, V_{H} or VH) and a heavy chain constant region. The heavy chain constant region comprises three domains, C_{H1}, C_{H2} and C_{H3}. Each light chain comprises a light chain variable region (abbreviated herein as LCVR, V_{L} or VL) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present technology include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}; (vi) V_{H}-C_{H2}-C_{H3}; (vii) V_{H}-CL; (viii) V_{L}-C_{H1}; (ix) V_{L}-C_{H2}, (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present technology may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present technology using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In specific embodiments, the antibody or antibody fragment for use in the method of the present technology may be a multispecific antibody, which may be specific for different epitopes of one target polypeptide or may contain antigen-binding domains specific for epitopes of more than one target polypeptide. An exemplary bi-specific antibody format that can be used in the context of the present technology involves the use of a first immunoglobulin (Ig) C_{H3} domain and a second Ig C_{H3} domain, wherein the first and second Ig C_{H3} domains differ from one another by at least one amino acid, and wherein at least one amino acid difference reduces binding of the bispecific antibody to Protein A as compared to a bi-specific antibody lacking the amino acid difference. In one embodiment, the first Ig C_{H3} domain binds Protein A and the second Ig C_{H3} domain contains a mutation that reduces or abolishes Protein A binding such as an H95R modification (by IMGT exon numbering; H43 5R by EU numbering). The second C_{H3} may further comprise an Y96F modification (by IMGT; Y436F by EU). Further modifications that may be found within the second C_{H3} include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present technology.

In some embodiments, the antibodies disclosed herein are human antibodies. The term “human antibody,” as used herein, is intended to include antibodies having variable and constant regions derived from human germline immunoglobulin sequences. The human antibodies featured in the present technology may in various embodiments nonetheless include amino acid residues not encoded by human germline immunoglobulin sequences (e.g., mutations introduced by random or site-specific mutagenesis in vitro or by somatic mutation in vivo), for example in the CDRs and in in some embodiments CDR3. However, the term human antibody, as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The present technology encompasses in various embodiments antibodies having one or more mutations in the hinge, C_{H2} or C_{H3} region which may be desirable, for example, in production, to improve the yield of the desired antibody form.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that specifically binds IL-6R, as used herein, includes antibodies that bind IL-6R or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. Specific binding can be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. Methods for determining whether two molecules specifically bind are well known in the art and include, for example, equilibrium dialysis, surface plasmon resonance, and the like. An antibody that specifically binds human IL-6R may, however, have cross-reactivity to other antigens, such as IL-6R molecules from other (non-human) species.

The term “surface plasmon resonance,” as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “KD” as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

A “neutralizing” or “blocking” antibody, as used herein, is intended to refer to an antibody that has binding to hIL-6R that results in inhibition of the biological activity of hIL-6. This inhibition of the biological activity of hIL-6 can be assessed by measuring one or more indicators of hIL-6 biological activity known to the art, such as hIL-6-induced cellular activation and hIL-6 binding to hIL-6R (see examples below).

The anti-IL-6R antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present technology includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework and/or CDR residues within the VH and/or VL domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, the antibodies of the present technology may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present technology.

The present technology also includes methods involving the use of anti-IL-6R antibodies comprising variants of any of the HCVR, LCVR, and/or CDR amino acid sequences disclosed herein having one or more conservative substitutions. For example, the present technology includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-IL-6R antibody, or antigen-binding fragment thereof, in various embodiments comprises a HCVR, LCVR, and/or CDRs comprising any of the amino acid sequences of the anti-IL-6R antibodies described in U.S. Pat. No. 7,582,298, incorporated herein by reference in its entirety.

In certain embodiments, the anti-IL-6R antibody or antigen-binding fragment thereof comprises the HCDRs of a HCVR comprising the amino acid sequence of SEQ ID NO: 2 and the LCDRs of a LCVR comprising the amino acid sequence of SEQ ID NO: 3. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 4; the HCDR2 comprises the amino acid sequence of SEQ ID NO: 5; the HCDR3 comprises the amino acid sequence of SEQ ID NO: 6; the LCDR1 comprises the amino acid sequence of SEQ ID NO: 7; the LCDR2 comprises the amino acid sequence of SEQ ID NO: 8; and the LCDR3 comprises the amino acid sequence of SEQ ID NO: 9.

In yet other embodiments, the anti-IL-6R antibody or antigen-binding fragment thereof comprises a heavy chain comprising the amino acid sequence of SEQ ID NO: 10 and a light chain comprising the amino acid sequence of SEQ ID NO: 11.

In another embodiment, the anti-IL-6R antibody or antigen-binding fragment thereof comprises a heavy chain comprising the amino acid sequence of SEQ ID NO: 9 and a light chain comprising the amino acid sequence of SEQ ID NO: 10. According to certain exemplary embodiments, the methods of the present disclosure comprise the use of the anti-IL-6R antibody referred to and known in the art as sarilumab, or a bioequivalent thereof.

The amino acid sequence of SEQ ID NO: 2 is

EVQLVESGGGLVQPGRSLRLSCAASRFTFDDYAMHWVRQAPGKGLEWVSG
ISWNSGRIGYADSVKGRFTISRDNAENSLFLQMNGLRAEDTALYYCAKGR
DSFDIWGQGTMVTVSS

The amino acid sequence of SEQ ID NO: 3 is

DIQMTQSPSSVSASVGDRVTITCRASQGISSWLAWYQQKPGKAPKLLIYG
ASSLESGVPSRFSGSGSGTDFTLTISSLQPEDFASYYCQQANSFPYTFGQ
GTKLEIK.

The amino acid sequence of SEQ ID NO: 4 is RFTFDDYA.

The amino acid sequence of SEQ ID NO: 5 is ISWNSGRI.

The amino acid sequence of SEQ ID NO: 6 is AKGRDSFDI.

The amino acid sequence of SEQ ID NO: 7 is QGISSW.

The amino acid sequence of SEQ ID NO: 8 is GAS.

The amino acid sequence of SEQ ID NO: 9 is QQANSFPYT.

The amino acid sequence of SEQ ID NO: 10 is

EVQLVESGGGLVQPGRSLRLSCAASRFTFDDYAMHWVRQAPGKGLEWVSG
ISWNSGRIGYADSVKGRFTISRDNAENSLFLQMNGLRAEDTALYYCAKGR
DSFDIWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
DPPSRELTKNQVSLTCLVKGFYPSDIAVEWESNGQPENNYKTTPPVLDSD
GSFFLYSKLTVDKSRWQQGNVFSCSVMHEALHNHYTQKSLSLSPGK.

The amino acid sequence of SEQ ID NO: 11 is

DIQMTQSPSSVSASVGDRVTITCRASQGISSWLAWYOOKPGKAPKLLIYG
ASSLESGVPSRFSGSGSGTDFTLTISSLQPEDFASYYCQQANSFPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In other specific embodiments, the antibody is sarilumab (SAR153191).

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The present technology in certain embodiments relates to methods comprising administering to the subject an antibody which comprises the heavy chain variable region comprising sequence SEQ ID NO:2 and the light chain variable region comprising sequence SEQ ID NO:1.

The disclosure provides pharmaceutical compositions comprising such antibody, and methods of using these compositions.

DMARDs

Disease modifying antirheumatic drugs (DMARDs) are drugs defined by their use in rheumatoid arthritis to slow down disease progression.

DMARDs have been classified as synthetic (sDMARD) and biological (bDMARD). Synthetic DMARDs include non-exhaustively methotrexate, sulfasalazine, leflunomide, and hydroxychloroquine. Biological DMARDs include non-exhaustively adalimumab, golimumab, etanercept, abatacept, infliximab, rituximab, and tocilizumab.

According to the compositions and methods of the disclosure, DMARDs can be administered as follows. Methotrexate can be administered from 10 to 25 mg per week orally or intramuscularly. In another embodiment, methotrexate is administered from 6 to 25 mg/week orally or intramuscularly for subjects who are from the Asia-Pacific region or who are descended from people who are from the Asia-Pacific region. The Asia-Pacific region includes Taiwan, South Korea, Malaysia, Philippines, Thailand and India. In certain embodiments, methotrexate is administered at between 6 and 12, 10 and 15, 15 and 20 and 20 and 25 mg per week. In other embodiments, methotrexate is administered at 6, 7, 8, 9, 10, 11, 12, 13, 14, 15, 16, 17, 18, 19, 20, 21, 22, 23, 24 or 25 mg per week. Leflunomide can be administered from 10 to 20 mg orally daily. In certain embodiments, leflunomide can be administered at between 10 and 12, 12 and 15, 15 and 17 and 18 and 20 mg per day. In other embodiments, leflunomide is administered at 10, 11, 12, 13, 14, 15, 16, 17, 18, 19 or 20 mg per day. Sulfasalazine can be administered from 1000 to 3000 mg orally daily. In certain embodiments, sulfasalazine can be administered at between 1000 and 1400, 1400 and 1800, 1800 and 2200, 2200 and 2600, and 2600 and 3000 mg per day. In other embodiments, sulfasalazine is administered at 1000, 1100, 1200, 1300, 1400, 1500, 1600, 1700, 1800, 1900, 2000, 2100, 2200, 2300, 2400, 2500, 2600, 2700, 2800, 2900 or 3000 mg per day. Hydroxychloroquine can be administered from 200 to 400 mg orally daily. In certain embodiments, hydroxychloroquine can be administered at between 200 and 240, 240 and 280, 280 and 320, 320 and 360 and 360 and 400 per day. In other embodiments, hydroxychloroquine can be administered at 200, 210, 220, 230, 240, 250, 260, 270, 280, 290, 300, 310, 320, 330, 340, 350, 360, 370, 380, 390 or 400 mg per day.

### Therapeutic Administration and Formulations

The methods described herein comprise administering a therapeutically effective amount of an anti-hIL-6R antibody. In some embodiments, the methods further comprise administering a DMARD to a subject. As used herein, the phrase “therapeutically effective amount” means a dose of anti-hIL-6R antibody and/or a DMARD that results in a detectable improvement in one or more symptoms associated with rheumatoid arthritis or which causes a biological effect (e.g., a decrease in the level of a particular biomarker) that is correlated with the underlying pathologic mechanism(s) giving rise to the condition or symptom(s) of rheumatoid arthritis. For example, a dose of anti-hIL-6R antibody and/or one or more DMARDs which causes an improvement in any of the following symptoms or conditions is deemed a “therapeutically effective amount”: chronic disease anemia, fever, depression, fatigue, rheumatoid nodules, vasculitis, neuropathy, scleritis, pericarditis, Felty's syndrome and/or joint destruction.

The efficacy of the antibody for treating rheumatoid arthritis is typically measured using the standard methods in the field, commonly used by the clinicians and the rheumatologists, for example the DAS-28 and American College of Rheumatism (ACR) parameters, for example the DAS-28 ESR, ACR20, ACR50 and ACR70 parameters. For example, a 20 (ACR20), 50 (ACR50) or 70% (ACR70) improvement from baseline can be used to show detectable improvement. Another measure used is the clinical disease activity index (CDAI).

The improvement of the physical function of the subjects suffering from rheumatoid arthritis is in various embodiments measured using the standard methods in the field, commonly used by the clinicians and the rheumatologists, namely the HAQ-DI parameter.

The improvement of the quality of life of the subjects suffering from rheumatoid arthritis is typically measured using the standard methods in the field, commonly used by the clinicians and the rheumatologists, for example the SF36 parameter, and in some embodiments the SF-36 PCS parameter.

The baseline (also referred to herein as “BL”) is defined as the score obtained by the subject before being administered with the antibody according to the present technology.

Change from baseline is defined as the difference existing between the score obtained by the subject at baseline and the score obtained by the subject after being administered with the antibody according to the present technology, for example measured at least 24 weeks after the first administration of the antibody, including 24 weeks after the first administration of the antibody.

DAS28-ESR

The disease activity score (DAS28) can be used to show detectable improvement. DAS28 is a composite score of tender joints count based on 28 joints, a swollen joints count based on 28 joints, a general health assessment and a marker of inflammation which can be assessed by measuring C-reactive protein (CRP) levels. The disease response can be presented using the European League against Rheumatism (EULAR) response criteria. A good response by these criteria is an improvement of greater than 1.2 in DAS28 score with a present score of greater than or equal to 3.2. A moderate response is an improvement of greater than 0.6 but less than or equal to 1.2 in DAS28 score and a present score of greater than 3.2. Non-response is an improvement of less than 0.6 in DAS28 score and a present score of greater than 5.1. DAS28 remission is a DAS28 score of less than 2.6.

DAS28 is a composite score that includes 4 variables:
Tender Joints Count (based on 28 joints: shoulder (n=2), elbow (n=2), wrist (n=2), metacarpophalangeal (n=10), interphalangeal of thumb (n=2), proximal interphalangeal (n=8), knee (n=2))
Swollen Joints count (based on 28 joints: shoulder (n=2), elbow (n=2), wrist (n=2), metacarpophalangeal (n=10), interphalangeal of thumb (n=2), proximal interphalangeal (n=8), knee (n=2))
General health assessment (GH) by the subject assessed from the ACR RA core set questionnaire (subject global assessment) in 100 mm visual analogue scale (VAS)
Marker of inflammation assessed by the CRP in mg/L or ESR in mm/hr.

It is a continuous measure allowing for measurement of absolute change in disease activity and percentage improvement. The DAS28-ESR can be calculated using the following formula:

DAS28-ESR=0.56×\sqrt{28TJC}+0.28×\sqrt{28TJC}+0.70×Ln[ESR(mm/h)]+0.014×GH(VAS)

The DAS28-ESR score provides a number indicating the current disease activity of the RA. A DAS28-ESR score above 5.1 means high disease activity, whereas a DAS28-ESR score below 3.2 indicates low disease activity and a DAS28-ESR score below 2.6 means disease remission.

When calculating the 28TJC and 28SJC, with individual missing joint scores (the ‘replaced or fused’ joints are not taken into consideration for the swelling or tenderness) imputed as the mean of the scored joints, the tender/swollen joint counts after imputation are as follows:

28TJC/28SJC=sum (scored tender/swollen joints)*(number of joints in the full joint set/number of scored tender/swollen joints). The number of joints in the full joint set is defined as (28−number of replaced or fused joints) and the scored joints refer to those with an answer (0—no pain, 1—pain).

If the subject answers that he or she is experiencing no pain (i.e., ESR=0), then for purposes of calculating the DAS-ESR score one should instead insert the value ESR=1 for the purpose of the DAS28-ESR calculation to enable a non-missing score.

DAS28-ESR is considered as missing if one of the components is missing.

Visual Analog Score (VAS)

The VAS is a measure to assess subject-related rheumatoid arthritis severity. Subject makes a vertical mark through each of two lines which best describes the amount of pain due to rheumatoid arthritis. Range from no pain to most severe pain.

ACR20

To be classified as an ACR20 responder, a subject must achieve 20% improvement compared with baseline, in both TJC and SJC, as well as 20% improvement in at least 3 out of the 5 remaining ACR components: physician's global assessment of disease activity, subject's global assessment of disease activity, pain, HAQ-DI, and CRP.

ACR50

ACR50 is defined as the event of achieving at least 50% improvement in both TJC and SJC, and at least 50% improvement in at least 3 out of the 5 remaining ACR components.

ACR70

ACR70 is defined as the event of achieving at least 70% improvement in both TJC and SJC and at least 70% improvement in at least 3 out of the 5 remaining ACR components.

The 7 ACR components assessing the signs and symptoms of RA are defined below (A-G):

A) Tender Joint Count (TJC)

A total of 68 joints are assessed for tenderness. The 68 joints to be examined for tenderness are: temporomandibular (n=2), sternoclavicular (n=2), acromioclavicular (n=2), shoulder (n=2), elbow (n=2), wrist (n=2), metacarpophalangeal (n=10), interphalangeal of thumb (n=2), distal interphalangeal (n=8), proximal interphalangeal (n=8), hip (n=2), knee (n=2), ankle mortise (n=2), ankle tarsus (n=2), metatarsophalangeal (n=10), interphalangeal of great toe (n=2), and proximal/distal interphalangeal of the toes (n=8).

A formal count of the joints is performed by a trained assessor. Joint tenderness is defined as pain induced by the pressure of the joints, exerted by the assessor's thumb and index finger. The assessor classifies each joint as painful (yes/no) and swollen (yes/no). A score of 0/1 is given to each tender joint with 0 representing no pain and 1 representing pain. The tender joint count ranges from 0 to 68 where 0 is considered the best and 68 the worst.

B) Swollen Joint Count (SJC)

The 66 joints to be examined for swelling are the same as those examined for tenderness, except the hip joints are not included. A formal count of the joints is performed by a trained assessor. The assessor classifies each joint as swollen (yes/no). A score of 0/1 is given to each swollen joint with 0 representing no swollen and 1 representing a swollen joint. The swollen joint count ranges from 0 to 66 where 0 is considered the best and 66 the worst.

C) Physician's Global Assessments of Disease Activity

Physician's global assessments of the subject's current disease activity is assessed on an anchored 100 mm horizontal VAS where 0 is considered the best disease activity (no disease activity) and 100 the worst (most disease activity).

D) Subject's Global Assessments of Disease Activity

Subject's global assessments of their current disease activity is rated on an anchored 100 mm horizontal VAS where 0 is considered the best disease activity (no disease activity) and 100 the worst (most disease activity).

E) Subject's Assessment of Pain

Subjects are requested to indicate their pain intensity due to their RA using a 100 mm horizontal VAS where 0 is considered “No pain” and 100 “the worst pain you can imagine”.

F) Subject's Assessment of Physical function—Health Assessment Questionnaire Disease Index (HAQ-DI)

The HAQ-DI is a standardized questionnaire developed for use in RA. The HAQ-DI, with the past week as the time frame, focuses on whether the respondent “is able to . . . ” do the activity and covers 8 categories in 20 items: dressing and grooming, arising, eating, walking, hygiene, reach, grip and activities, for which there are at least 2 questions by category. The 4 responses for the HAQ-DI questions are graded as follows: without any difficulty=0; with some difficulty=1; with much difficulty=2; and unable to do=3. To calculate the Standard HAQ-DI Score (With Aids/Devices), there are 3 steps:

1. Sum the 8 category scores by using the highest sub-category score from each category. For example, in the category EATING there are 3 sub-category items. A subject responds with a 1, 2, and 0, respectively; the category score is 2.

2. Adjust for use of aids/devices and/or help from another person when indicated.
Adjust the score for a category by increasing a 0 or a 1 to a 2.
If a subject's highest score for that sub-category is a 2 it remains a 2, and if a 3, it remains a 3.
The data entered at field “Other specify” are not used for score adjustment.

3. Divide the summed category scores by the number of categories answered (must be a minimum of 6) to obtain an HAQ-DI score of 0 to 3 (3=worst functioning).

An HAQ-DI score cannot be calculated validly when there are scores for less than 6 of the 8 categories. HAQ-DI scoring ranges between 0 and 3. A high HAQ-DI score has been found to be a strong predictor of morbidity and mortality in RA. A 0.22 unit difference is considered clinically meaningful.

G) The level of an acute phase reactant measured by CRP

High sensitivity CRP is assessed centrally. Since CRP levels are directly correlated with Interleukin 6 (IL-6) receptor activity, it is expected that active dose regimens has a dramatic lowering effect on CRP levels. Therefore, during the study, post-dosing CRP remains blinded to the investigators, the sponsor and the subjects.

The ACR components are further described in Table 1.

TABLE 1
ACR components

| ACR components | Range | Direction |
|---|---|---|
| TJC | 0 to 68 | Lower is better |
| SJC | 0 to 66 | Lower is better |
| Pain VAS | 0 to 100 | Lower is better |
| Patient global VAS | 0 to 100 | Lower is better |
| Physician global VAS | 0 to 100 | Lower is better |
| HAQ-DI | 0 to 3 | Lower is better |
| CRP (mg/dL) | >0 | Lower is better |

TJC: Total Joint Counts, SJC: Swollen Joint Counts, VAS: Visual Analog Scale

SF-36 V2

The QualityMetric's SF-36v2® Health Survey is a multi-purpose, short-form health survey with 36 questions. It yields scores for eight domains (Physical Functioning, Role-Physical, Bodily pain, General health, Vitality, Social Functioning, Role-Emotional, and Mental Health, where each domain is scored from 0 to 100 and where higher scores indicate better health and well-being), as well as two summary measures of physical and mental health: the physical component summary (PCS) and mental component summary (MCS).

The scoring process is summarized below:

1. Enter item response data.

2. Recode item response values.

3. Determine health domain scale raw scores.

4. Transform health domain scale raw scores to 0 to 100 scores.

5. Transform health domain scale 0 to 100 to normal-based scores.

6. Score PCS and MCS measures.

The following Table 2 shows the construction and summary measures of the SF-36 scales:

TABLE 2
SF-36 V2 measurement model

| Summary | | |
|---|---|---|
| Measures | Scales | Items |
| PCS* | Physical Functioning (PF) | 3a, 3b, 3c, 3d, 3e, |
| | | 3f, 3g, 3h, 3i, 3j |
| | Role-Physical (RP) | 4a, 4b, 4c, 4d |
| | Bodily Pain (BP) | 7,8 |
| | General Health (GH) | 1, 11a, 11b, 11c, 11d |
| MCS) | Vitality (VT) | 9a, 9e, 9g, 9i |
| | Social Functioning (SF) | 6, 10 |
| | Role-Emotional (RE) | 5a, 5b, 5c |
| | Mental Health (MH) | 9b, 9c, 9d, 9f, 9h |

*MCS and PCS derived from the eight scales (Ware, JE. et al. 1994 “SF-36 Physical and Mental Health Summary Scales: A Users’ Manual”. Boston: The Health Institute)

The Abbreviated Item Content is as follows:

3a Vigorous activities, such as running, lifting heavy objects, or participating in strenuous sports;

3b Moderate activities, such as moving a table, pushing a vacuum cleaner, bowling, or playing golf;

3c Lifting or carrying groceries;

3d Climbing several flights of stairs;

3e Climbing one flight of stairs;

3f Bending, kneeling, or stooping;

3g Walking more than a mile;

3h Walking several hundred yards;

3i Walking one hundred yards;

3j Bathing or dressing oneself;

4a Cut down the amount of time one spent on work or other activities;

4b Accomplished less than you would like;

4c Limited in kind of work or other activities;

4d Had difficulty performing work or other activities (e.g., it took extra effort);

7 Intensity of bodily pain;

8 Extent pain interfered with normal work;

1 Is your health: excellent, very good, good, fair, poor;

11a Seem to get sick a little easier than other people;

11b As healthy as anybody I know;

11c Expect my health to get worse;

11 d Health is excellent;

9a Feel full of life;

9e Have a lot of energy;

9g Feel worn out;

9i Feel tired;

6 Extent health problems interfered with normal social activities;

10 Frequency health problems interfered with social activities;

5a Cut down the amount of time spent on work or other activities;

5b Accomplished less than you would like;

5c Did work or other activities less carefully than usual;

9b Been very nervous;

9c Felt so down in the dumps that nothing could cheer you up;

9d Felt calm and peaceful;

9f Felt downhearted and depressed; and

9h Been happy.

The PCS and MCS summary measure scores are computed if at least 50% of the component scales are available. The scale scores are computed if at least 50% of items are available within the corresponding scale. The missing items are imputed by the mean of available items.

General Scoring information

Items and scales are scored in 3 steps:
Step 1. Item recoding, for the 10 items that require recoding,
Step 2. Computing scale scores by summing across items in the same scale (raw scale scores); and,
Step 3. Transforming raw scale scores to a 0-100 scale (transformed scale).

### Item Recoding

All 36 items should be checked for out-of-range values prior to assigning the final item value. All out-of-range values should be recoded as missing data.
The following tables show the recoding of response choice.

### How to Treat Missing Data

A scale score is calculated if a respondent answered at least half of the items in a multi-item scale (or half plus one in the case of scales with an odd number of items).

The recommended algorithm substitutes a person-specific estimate for any missing item when the respondent answered at least 50 percent of the items in a scale. A psychometrically sound estimate is the average score, across completed items in the same scale, for that respondent. For example, if a respondent leaves one item in the 5-item mental Health scale blank, one must substitute the respondent's average score (across the 4 completed mental health items) for that one item. When estimating the respondent's average score, use the respondent's final item values.

### Computing Raw Scale Scores

After item recoding, including handling of missing data, a raw score is computed for each scale. This score is a simple algebraic sum of responses for all items in that scale.

If the respondent answered at least 50% of the items in a multi-items scale, the score can be calculated. If the respondent did not answer at least 50% of the items, the score for that scale should be set to missing.

### Transformation of the Scale Scores

The next step involves transforming each raw score to a 0 to 100 scale using the following formula:

Transformed scale=[(actual raw score−lowest possible raw score)/possible raw score range]×100

This transformation converts the lowest and highest possible scores to zero and 100, respectively.

TABLE 3
SF-36 V2 raw scores of eight domains

| | Lowest and Highest | raw score |
|---|---|---|
| Scale | possible raw scores | range |
| Physical Functioning | 10 and 30 | 20 |
| Role-Physical | 4 and 20 | 16 |
| Bodily pain | 2 and 12 | 10 |
| General health | 5 and 25 | 20 |
| Vitality | 4 and 20 | 16 |
| Social Functioning | 2 and 10 | 8 |
| Role-Emotional | 3 and 15 | 12 |
| Mental Health | 5 and 25 | 20 |

The score of each of the 36 items is collected in CRF (case report form). Then, a SAS (Statistical Analysis System) code (e.g. the one provided by the QualityMetric survey) is used to calculate the eight scales, the two summary measure scores and the standardized summary scores.

The PCS and MCS summary measure scores are computed if at least 50% of the component scales are available. The scale scores are computed if at least 50% of items are available within the corresponding scale. The missing items are imputed by the mean of available items.

Change from BL in SF-36 scores (physical component summary score and mental component summary score as well as the eight domains) is then analyzed.

### SF-36 V2 Scoring

General Scoring Information

Items and scales are scored in 3 steps (as instructed by the QualityMetric survey manual):
Step 1. Item recoding, for the 10 items that require recoding
Step 2. Computing scale scores by summing across items in the same scale (raw scale scores); and
Step 3. Transforming raw scale scores to a 0-100 scale (transformed scale)
Step 5: Compute Z-Scores
Step 6: Convert Z-score to Norm Based scores for domains
PCS: Compute aggregate PCS score using a specific weighted formula, convert this into a Norm based score
MCS: Compute aggregate MCS score using a specific weighted formula, convert this into a Norm based score

### Item Recoding

All 36 items should be checked for out-of-range values prior to assigning the final item value. All out-of-range values should be recoded as missing data.

### How to Treat Missing Data

A scale score is calculated if a respondent answered at least half of the items in a multi-item scale (or half plus one in the case of scales with an odd number of items).

The recommended algorithm substitutes a person-specific estimate for any missing item when the respondent answered at least 50 percent of the items in a scale. A psychometrically sound estimate is the average score, across completed items in the same scale, for that respondent. For example, if a respondent leaves one item in the 5-item mental Health scale blank, one must substitute the respondent's average score (across the 4 completed mental health items) for that one item. When estimating the respondent's average score, use the respondent's final item values.

### Computing Raw Scale Scores

After item recoding, including handling of missing data, a raw score is computed for each scale. This score is a simple algebraic sum of responses for all items in that scale.

If the respondent answered at least 50% of the items in a multi-items scale, the score can be calculated. If the respondent did not answer at least 50% of the items, the score for that scale should be set to missing.

### Transformation of the Scale Scores

The next step involves transforming each raw score to a 0 to 100 scale using the following formula:

Transformed scale=[(actual raw score−lowest possible raw score)/possible raw score range]×100.

This transformation converts the lowest and highest possible scores to zero and 100, respectively.

Clinical Disease Activity Index (CDAI)

The clinical disease activity index (CDAI) is an established instrument to measure disease activity in rheumatoid arthritis (RA). The CDAI is composite index calculated as the sum of five outcome parameters:

TEN28, tender joint count based on a 28-joint assessment (0-28);

SWJ28, swollen joint count based on a 28-joint assessment (0-28);

“PGAVAS,” patient global assessment of disease activity on a visual analogue scale (VAS) (0-10); and

“INVVAS,” investigator global assessment of disease activity on a visual analogue scale (VAS) (0-10).

It is calculated by the formula: CDAI=TEN28+SWJ28+PGAVAS+INVVAS.

CDAI does not include the C-Reactive Protein. An immediate scoring is, therefore, possible in a clinical trial.

The CDAI score is interpreted as follows:

0.0-2.8—Remission;

2.9-10.0—Low Activity;

10.1-22.0—Moderate Activity;

22.1-76.0—High Activity.

In accordance with the methods of the present technology, a therapeutically effective amount of anti-hIL-6R antibody that is administered to the subject will vary depending upon the age and the size (e.g., body weight or body surface area) of the subject as well as the route of administration and other factors well known to those of ordinary skill in the art. In certain embodiments, the dose of anti-hIL-6R antibody administered to the subject is from about 10 mg to about 500 mg. For example, the present technology includes methods wherein about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300, about 325 mg, about 350 mg, about 375 mg, about 400 mg, about 425 mg, about 450 mg, about 475 mg, about 500 mg, or more of anti-hIL-6R antibody is administered to the subject per week.

In one embodiment, the hIL-6R antibody is administered at 100-150 mg per week. In another embodiment, the hIL-6R antibody is administered at 100-200 mg per ever two weeks. In other embodiments, the hIL-6R antibody is administered at about 100 or about 150 mg per week. In other embodiments, the hIL-6R antibody is administered at about 100, 150 or 200 mg per every two weeks. “Once every two weeks” has the same meaning as “q2w” or “once per two weeks”, i.e. that the antibody is administered once in a two-week period of time. According to certain embodiments, the antibody is administered subcutaneously.

In certain embodiments, the antibody is administered at about 100 mg, 150 mg or about 200 mg once every two weeks. In this context, “about” refers to an amount within 5% of the stated amount. For example, “about 100 mg” is a range of between 95 and 105 mg. According to certain embodiments, the antibody is administered subcutaneously.

The amount of anti-hIL-6R antibody that is administered to the subject may be expressed in terms of milligrams of antibody per kilogram of subject body weight (i.e., mg/kg). For example, the methods of the present technology include administering an anti-hIL-6R antibody to a subject at a daily dose of about 0.01 to about 100 mg/kg, about 0.1 to about 50 mg/kg, or about 1 to about 10 mg/kg of subject body weight.

In various embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
between about 100 mg/mL and about 200 mg/mL of the antibody.

In another embodiment, the antibody is administered as an aqueous buffered solution at pH 6.0 containing
about 21 mM histidine,
about 45 mM arginine,
about 0.2% (w/v) polysorbate 20,
about 5% (w/v) sucrose, and
at least about 130 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
about 131.6 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose; and
about 175 mg/mL of the antibody.

In other embodiments, the antibody is administered as an aqueous buffered solution at pH 6.0 containing
21 mM histidine,
45 mM arginine,
0.2% (w/v) polysorbate 20,
5% (w/v) sucrose, and
between 100 mg/mL and 200 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
at least 130 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
131.6 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose; and
175 mg/mL of the antibody.

The methods of the present technology include administering multiple doses of an anti-hIL-6R antibody to a subject over a specified time course. For example, the anti-hIL-6R antibody can be administered about 1 to 5 times per day, about 1 to 5 times per week, about 1 to 5 times per month or about 1 to 5 times per year. In certain embodiments, the methods of the present technology include administering a first dose of anti-hIL-6R antibody to a subject at a first time-point, followed by administering at least a second dose of anti-hIL-6R antibody to the subject at a second time-point. The first and second doses, in certain embodiments, may contain the same amount of anti-hIL-6R antibody. For instance, the first and second doses may each contain about 10 mg to about 500 mg, about 20 mg to about 300 mg, about 100 mg to about 200 mg, or about 100 mg to about 150 mg of the antibody. The time between the first and second doses may be from about a few hours to several weeks. For example, the second time-point (i.e., the time when the second dose is administered) can be from about 1 hour to about 7 weeks after the first time-point (i.e., the time when the first dose is administered). According to certain exemplary embodiments of the present technology, the second time-point can be about 1 hour, about 4 hours, about 6 hours, about 8 hours, about 10 hours, about 12 hours, about 24 hours, about 2 days, about 3 days, about 4 days, about 5 days, about 6 days, about 7 days, about 2 weeks, about 4 weeks, about 6 weeks, about 8 weeks, about 10 weeks, about 12 weeks, about 14 weeks or longer after the first time-point. In certain embodiments, the second time-point is about 1 week or about 2 weeks. Third and subsequent doses may be similarly administered throughout the course of treatment of the subject.

The present technology provides methods of using therapeutic compositions comprising anti-IL-6R antibodies or antigen-binding fragments thereof and/or one or more DMARDs. The therapeutic compositions of the present technology will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the weight of a subject to be administered, target disease, conditions, route of administration, and the like. Various delivery systems are known and can be used to administer the pharmaceutical composition of the present technology, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The hIL-6R antibody can be administered subcutaneously. The DMARD can be administered orally or intramuscularly.

The pharmaceutical composition can also be delivered in a vesicle, in particular a liposome (see Langer (1990) Science 249:1527-1533). In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The antibody is typically formulated as described herein and in international publication number WO2011/085158, incorporated herein by reference in its entirety.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the DMARD contained is generally about 5 to 3000 mg per dosage form in an oral unit dose depending on the specific DMARD used. The amount of the hIL-6R antibody contained is generally about 100 to 200 mg per subcutaneous dosage form.

In accordance with the methods disclosed herein, the anti-hIL-6R antibody (or pharmaceutical formulation comprising the antibody) can be administered to the subject using any acceptable device or mechanism. For example, the administration can be accomplished using a syringe and needle or with a reusable pen and/or autoinjector delivery device. The methods of the present technology include the use of numerous reusable pen and/or autoinjector delivery devices to administer an anti-hIL-6R antibody (or pharmaceutical formulation comprising the antibody). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present technology include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few.

The use of a microinfusor to deliver an anti-hIL-6R antibody (or pharmaceutical formulation comprising the antibody) to a subject is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) and/or viscous solutions.

In one embodiment, the antibody is administered with a prefilled syringe.

In another embodiment, the antibody is administered with a prefilled syringe containing a safety system. For example, the safety system prevents an accidental needlestick injury. In various embodiments, the antibody is administered with a prefilled syringe containing an ERIS™ safety system (West Pharmaceutical Services Inc.). See also U.S. Pat. Nos. 5,215,534 and 9,248,242, incorporated herein by reference in their entireties.

In another embodiment, the antibody is administered with an auto-injector. In various embodiments, the antibody is administered with an auto-injector featuring the PUSHCLICK™ technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

### Combination Therapies

The present technology includes methods of treating rheumatoid arthritis which comprise administering to a subject in need of such treatment an anti-hIL-6R antibody in combination with at least one additional therapeutic agent. Examples of additional therapeutic agents which can be administered in combination with an anti-hIL-6R antibody in the practice of the methods of the present technology include, but are not limited to DMARDs, and any other compound known to treat, prevent, or ameliorate rheumatoid arthritis in a human subject. Specific, non-limiting examples of additional therapeutic agents that may be administered in combination with an anti-hIL-6R antibody in the context of a method of the present technology include, but are not limited to methotrexate, sulfasalazine, hydroxychloroquine and leflunomide. In the present methods, the additional therapeutic agent(s) can be administered concurrently or sequentially with the anti-hIL-6R antibody. For example, for concurrent administration, a pharmaceutical formulation can be made which contains both an anti-hIL-6R antibody and at least one additional therapeutic agent. The amount of the additional therapeutic agent that is administered in combination with the anti-hIL-6R antibody in the practice of the methods of the present technology can be easily determined using routine methods known and readily available in the art.

The disclosure of the present technology provides for pharmaceutical compositions comprising any of the following:

A composition comprising between 100 and 150 mg of sarilumab (SA153191) and 10-25 mg of methotrexate.

A composition comprising between 100 and 200 mg of sarilumab (SA153191) and 10-25 mg of methotrexate.

A composition comprising between 100 and 150 mg of sarilumab (SA153191) and 6-25 mg of methotrexate.

A composition comprising between 100 and 200 mg of sarilumab (SA153191) and 6-25 mg of methotrexate.

A composition comprising between 100 and 150 mg of sarilumab (SA153191) and 10-20 mg of leflunomide.

A composition comprising between 100 and 200 mg of sarilumab (SA153191) and 10-20 mg of leflunomide.

A composition comprising between 100 and 150 mg of sarilumab (SA153191) and 1000-3000 mg of sulfasalazine.

A composition comprising between 100 and 200 mg of sarilumab (SA153191) and 1000-3000 mg of sulfasalazine.

A composition comprising between 100 and 150 mg of sarilumab (SA153191) and 200-400 mg of hydroxychloroquine.

A composition comprising between 100 and 200 mg of sarilumab (SA153191) and 200-400 mg of hydroxychloroquine.

The disclosure of the present technology provides for methods of improving symptoms associated with rheumatoid arthritis comprising any of the following:

A method comprising administering between 100 and 150 mg of sarilumab (SA153191) and 10-25 mg of methotrexate per week to a subject in need thereof.

A method comprising administering between 100 and 200 mg of sarilumab (SA153191) every two weeks and 10-25 mg of methotrexate per week to a subject in need thereof.

A method comprising administering between 100 and 150 mg of sarilumab (SA153191) and 6-25 mg of methotrexate per week to a subject in need thereof.

A method comprising administering between 100 and 200 mg of sarilumab (SA153191) every two weeks and 6-25 mg of methotrexate per week to a subject in need thereof.

A method comprising administering between 100 and 150 mg of sarilumab (SA153191) per week and 10-20 mg of leflunomide per day to a subject in need thereof.

A method comprising administering between 100 and 200 mg of sarilumab (SA153191) every two weeks and 10-20 mg of leflunomide per day to a subject in need thereof.

A method comprising administering between 100 and 150 mg of sarilumab (SA153191) per week and 1000-3000 mg of sulfasalazine per day to a subject in need thereof.

A method comprising administering between 100 and 200 mg of sarilumab (SA153191) every two weeks and 1000-3000 mg of sulfasalazine per day to a subject in need thereof.

A method comprising administering between 100 and 150 mg of sarilumab (SA153191) per week and 200-400 mg of hydroxychloroquine per day to a subject in need thereof.

A method comprising administering between 100 and 200 mg of sarilumab (SA153191) every two weeks and 200-400 mg of hydroxychloroquine per day to a subject in need thereof.

### Biomarkers

The present disclosure includes methods of treating rheumatoid arthritis by administering to a subject in need of such treatment a therapeutically effective amount of an antibody or antibody binding fragment thereof which specifically binds to hIL-6R and, optionally, a therapeutically effective amount of one or more DMARDs, wherein the level of one or more RA-associated biomarkers in the subject is modified (e.g., increased, decreased, etc., as the case may be) following administration. In a related aspect, the present technology includes methods for decreasing an RA-associated biomarker in a subject by administering to the subject a therapeutically-effective amount of an antibody or antigen-binding fragment thereof which specifically binds to hIL-6R and, optionally, a therapeutically effective amount of one or more DMARDs.

Examples of RA-associated biomarkers include, but are not limited to, e.g., high-sensitivity C-reactive protein (hsCRP), serum amyloid A (SAA), erythrocyte sedimentation rate (ESR), serum hepcidin, interleukin-6 (IL-6), and hemoglobin (Hb). As will be appreciated by a person of ordinary skill in the art, an increase or decrease in an RA-associated biomarker can be determined by comparing the level of the biomarker measured in the subject at a defined time point after administration of the anti-IL-6R antibody to the level of the biomarker measured in the subject prior to the administration (i.e., the “baseline measurement”). The defined time point at which the biomarker can be measured can be, e.g., at about 4 hours, 8 hours, 12 hours, 1 day, 2 days, 3 days, 4 days, 5 days, 6 days, 7 days, 8 days, 9 days, 10 days, 15 days, 20 days, 35 days, 40 days or more after administration of the anti-hIL-6R antibody.

According to certain embodiments of the present technology, a subject may exhibit a decrease in the level of one or more of hsCRP, SAA, ESR and/or hepcidin following administration of an anti-hIL-6R antibody to the subject. For example, at about week 12 following weekly administration of anti-hIL-6R antibody and, optionally, one or more DMARDs the subject may exhibit one or more of the following: (i) a decrease in hsCRP by about 40%, 45%, 50%, 55%, 60%, 65%, 70%, 75%, 80%, 85%, 90%, 95% or more; (ii) a decrease in SAA by about 40%, 45%, 50%, 55%, 60%, 65%, 70%, 75%, 80%, 85%, 90%, 95% or more; (iii) a decrease in ESR by about 15%, 20%, 25%, 30%, 35%, 40%, 45%, 50%, 55% or more; and/or (iv) a decrease in hepcidin by about 30%, 35%, 40%, 45%, 50%, 55%, 60%, 65%, 70%, 75% or more.

According to certain other embodiments of the present technology, a subject may exhibit an increase in the level of one or more of Hb or IL-6 following administration of an anti-hIL-6R antibody and, optionally, one or more DMARDs to the subject. For example, at about week 12 following weekly administration of anti-hIL-6R antibody and, optionally, one or more DMARDs the subject may exhibit one or more of the following: (v) an increase in Hb by about 0.5%, 1.0%, 1.5%, 2.0%, 2.5%, 3.0%, 3.5%, 4.0%, 4.5%, 5.0%, 5.5%, 6.0% or more; and/or (vi) an increase in IL-6 by about 100%, 150%, 200%, 250%, 300%, 350%, 400%, 450%, 500%, 550%, 600%, 650%, 700%, 750%, 800% or more.

The present technology includes methods for determining whether a subject is a suitable subject for whom administration of an anti-hIL-6R antibody would be beneficial. For example, if an individual, prior to receiving an anti-hIL-6R antibody and/or one or more DMARDs, exhibits a level of an RA-associated biomarker which signifies the disease state, the individual is therefore identified as a suitable subject for whom administration of an anti-hIL-6R antibody would be beneficial. According to certain exemplary embodiments, an individual may be identified as a good candidate for anti-hIL-6R/DMARD therapy if the individual exhibits one or more of the following: (i) a level of hsCRP greater than about 4 mg/L (e.g., about 4.5 mg/L, about 5.0 mg/L, about 5.5 mg/L, about 6.0 mg/L, about 7.0 mg/L, about 10.0 mg/L, about 15.0 mg/L, about 20.0 mg/L, or more); (ii) a level of SAA greater than about 3800 ng/mL (e.g., about 4000 ng/mL, 4500 ng/mL, about 5000 ng/mL, about 5500 ng/mL, about 6000 ng/mL, about 10,000 ng/mL, about 20,000 ng/mL, about 25,000 ng/mL, about 30,000 ng/mL, about 35,000 ng/mL, about 40,000 ng/mL, about 45,000 ng/mL, or more); (iii) an ESR greater than about 15 mm/hour (e.g., about 16 mm/hour, about 17 mm/hour, about 18 mm/hour, about 19 mm/hour, about 20 mm/hour, about 21 mm/hour, about 22 mm/hour, about 25 mm/hour, about 30 mm/hour, about 35 mm/hour, about 40 mm/hour, about 45 mm/hour, about 50 mm/hour, or more); and/or (iv) a level of hepcidin greater than about 60 ng/mL (e.g., about 62 ng/mL, about 64 ng/mL, about 68 ng/mL, about 70 ng/mL, about 72 ng/mL, about 74 ng/mL, about 76 ng/mL, about 78 ng/mL, about 80 ng/mL, about 82 ng/mL, about 84 ng/mL, about 85 ng/mL, about 90 ng/mL, about 95 ng/mL, about 100 ng/mL, about 105 ng/mL, or more). Additional criteria, such as other clinical indicators of RA, may be used in combination with any of the foregoing RA-associated biomarkers to identify an individual as a suitable candidate for anti-hIL-6R therapy.

### Subject Populations

According to the present technology, “subject” means a human subject or human patient.

In certain embodiments, the methods and compositions described herein are administered to specific subject populations. These populations include subjects that have previously been treated for rheumatoid arthritis with treatment regimens other than an anti-hIL-6R antibody. These treatment regimens include DMARDs such as anti-TNF-α therapy (TNFi), e.g., biologic anti-TNF-α treatment regimens, and/or JAK-inhibitor (JAKi) therapy. Biologic anti-TNF-α antagonists include etanercept, infliximab, adalimumab, golimumab and certolizumab pegol. JAK-inhibitors include baricitinib, tofacitinib, and upadacitinib. These treatment regimens also include other DMARD therapy in the absence of anti-hIL-6R antibody. DMARDs used in this therapy include methotrexate, sulfasalazine, hydroxychloroquine and leflunomide. The DMARDs may be administered alone or in combination with another therapy that is not an anti-hIL-6R antibody. In a specific embodiment, the previous treatment regimen was methotrexate. In certain embodiments, the subject has been administered both anti-TNF-α and other DMARD therapies. In a specific embodiment, the previous treatment regimen was a JAKi. In certain embodiments, the subject has been administered both JAKi and other DMARD therapies.

According to the present technology, a subject who is considered “ineffectively treated” is a subject who in various embodiments either has shown to be intolerant to the one or more previous treatment regimens, and/or a subject who has shown an inadequate response to the one or more previous treatment regimen, typically a subject who is still considered to present with, or to have, active rheumatoid arthritis despite the previous one or more treatment regimen administered. The “Active rheumatoid arthritis” is typically defined as:
at least 6 of 66 swollen joints and 8 of 68 tender joints, as counted by the physician in a typical quantitative swollen and tender joint count examination,
High sensitivity C-reactive protein (hs-CRP) ≥8 mg/L or ESR ≥28 mm/H
DAS28ESR >5.1.

In one embodiment, the subject, who was previously ineffectively treated for rheumatoid arthritis by administering at least one DMARD different from the antibody, is a subject who was previously ineffectively treated for rheumatoid arthritis by administering a DMARD selected from the group consisting of methotrexate, sulfasalazine, leflunomide, and hydroxychloroquine. In various embodiments, the DMARD is methotrexate.

In another embodiment, the subject, who was previously ineffectively treated for rheumatoid arthritis by administering one or more DMARD different from the antibody, is a subject who had an inadequate response or intolerance to methotrexate.

According to the present technology, for those subjects previously ineffectively treated for rheumatoid arthritis by administering one or more DMARD different from the antibody, the one or more DMARD is/are not administered anymore to the subject, and the antibody is in various embodiments administered alone, in monotherapy to the subject. According to the present technology, “monotherapy” means that the subject receiving the antibody is not administered with any other DMARD (or other treatment regimen) in course of administration with the antibody.

In one embodiment, the subject, who was previously ineffectively treated for rheumatoid arthritis by administering treatment regimens other than the combination of an anti-hIL-6R antibody and, optionally, one or more DMARDs, is a subject who was previously ineffectively treated for rheumatoid arthritis by administering an anti-TNF-α treatment regimen.

In one embodiment, the subject, who was previously ineffectively treated for rheumatoid arthritis by administering treatment regimens other than the combination of an anti-hIL-6R antibody and, optionally, one or more DMARDs, is a subject who was previously ineffectively treated for rheumatoid arthritis by administering a JAKi treatment regimen.

In another embodiment, the subject, who was previously ineffectively treated for rheumatoid arthritis by administering one or more treatment regimens different from the antibody, is a subject who had an inadequate response or intolerance to an anti-TNF-α treatment regimen. In another embodiment, the subject, who was previously ineffectively treated for rheumatoid arthritis by administering one or more treatment regimens different from the antibody, is a subject who had an inadequate response or intolerance to a JAKi treatment regimen.

According to the present technology, for those subjects previously ineffectively treated for rheumatoid arthritis by administering one or more treatment regimen different from the antibody, the one or more treatment is/are not administered anymore to the subject, and the antibody is in various embodiments administered alone, in monotherapy to the subject.

In various embodiments, the subject is intolerant to the DMARD and/or other treatment regimen due to one or more physical reactions, conditions or symptoms from the treatment with the DMARD or other treatment regimen. Physical reactions, conditions or symptoms can include allergies, pain, nausea, diarrhea, azotemia, bleeding of the stomach, intestinal bleeding, canker sores, decreased blood platelets, perforation of the intestine, bacterial infection, inflammation of gums or mouth, inflammation of the stomach lining or intestinal lining, bacterial sepsis, stomach ulcer, intestinal ulcer, sun sensitive skin, dizziness, loss of appetite, low energy, and vomiting. In certain embodiments, intolerance can be determined by the subject or by a medical professional upon examination of the subject. In various embodiments, the DMARD is selected from the group consisting of methotrexate, sulfasalazine, leflunomide, and hydroxychloroquine. In certain embodiments, the DMARD is methotrexate. In certain embodiments, the DMARD is an anti-TNFα treatment regimen. In certain embodiments, the DMARD is a JAKi treatment regimen.

In other embodiments, the subject suffers from diminishment in quality of life due to RA. In certain embodiments, subjects suffering from diminishment in quality of life due to RA score as more severe than average on a metric selected from Change From Baseline in European Quality of Life-5 Dimension 3 Level (EQ-5D-3L), Change From Baseline in Rheumatoid Arthritis Impact of Disease (RAID), Work Days Missed Due to Arthritis, Work Productivity Reduced by ≥50% Due to Arthritis, Rate of Arthritis Interference With Work Productivity, House Work Days Missed Due to Arthritis, Days With Household Work Productivity Reduced by ≥50% Due to Arthritis, Days With Family/Social/Leisure Activities Missed Due to Arthritis, Days With Outside Help Hired Due to Arthritis, Rate of RA Interference With Household Work Productivity, Morning Stiffness VAS, Individual ACR Component—TJC and SJC, Individual ACR Component—Physician Global VAS, Participant Global VAS and Pain VAS, and Individual ACR Component—ESR Level. In other embodiments, the subject has a more severe than average HAQ-DI or DAS-28 score before starting treatment.

In certain embodiments, subjects who score as more severe than average on one or more metrics listed above have a score that is more severe than the baseline value for the metric. In various embodiments, a subject having a score of baseline value or more severe than baseline value on one or more metrics listed above, after receiving treatment indicates that the subject is an inadequate responder to the treatment. In other embodiments, a subject having a score more severe than baseline value on one or more metrics listed above, after receiving treatment indicates that the subject is an inadequate responder to the treatment.

In certain embodiments, the subjects who have scores for metrics that are more severe than the baseline value for the metric listed above have scores that are at least 10, 20, 30, 40, 50, 60, 70, 80, 90 or 100% more severe than baseline.

The therapies may be performed sequentially in any order or simultaneously. In certain embodiments, these therapies have been received by the subject within 2 years prior to receiving the combination of an anti-hIL-6R antibody and one or more DMARDs. In other embodiments, these therapies have been received within 1, 2, 3, 4, 5, 6, 7, 8, 9 or 10 years prior to receiving the combination of an anti-hIL-6R antibody and one or more DMARDs.

In certain embodiments, the methods and compositions described herein are administered to specific subject populations that have received one or more of the treatment regimens described above wherein these treatments have not been effective. As used herein, a treatment has not been effective when one or more dose does not result in a detectable improvement in one or more symptoms associated with rheumatoid arthritis or which does not cause a biological effect (e.g., a decrease in the level of a particular biomarker) that is correlated with the underlying pathologic mechanism(s) giving rise to the condition or symptom(s) of rheumatoid arthritis. For example, a treatment which does not cause an improvement in any of the following symptoms or conditions is deemed ineffective: chronic disease anemia, fever, depression, fatigue, rheumatoid nodules, vasculitis, neuropathy, scleritis, pericarditis, Felty's syndrome and/or joint destruction.

A detectable improvement can also be detected using the American College of Rheumatism (ACR) rheumatoid arthritis classification criteria. For example, a 20 (ACR20), 50 (ACR50) or 70% (ACR70) improvement from baseline can be used to show detectable improvement. A detectable improvement can also be shown by measuring an improvement in any of the components of the DAS28 score.

The present invention is further illustrated by the following examples which should not be construed as further limiting. The contents of the figures and all references, patents and published patent applications cited throughout this application are expressly incorporated herein by reference for all purposes.

Furthermore, in accordance with the present invention there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

This example shows that treatment with an antibody of the present technology is effective in treating RA subjects that were ineffectively treated with JAKi.

Methods:

348 RA subjects were treated with sarilumab. RA subjects were selected at physician discretion and treated according to the drug label. An interim analysis was performed at 12 weeks.

Results:

265 subjects in the study had post-baseline data up to 12 weeks.

The mean age of the subjects analyzed was 58.6 years (24-83); 232 out of 304 patients (76.3%) are female. The mean disease duration was 10.3 years.

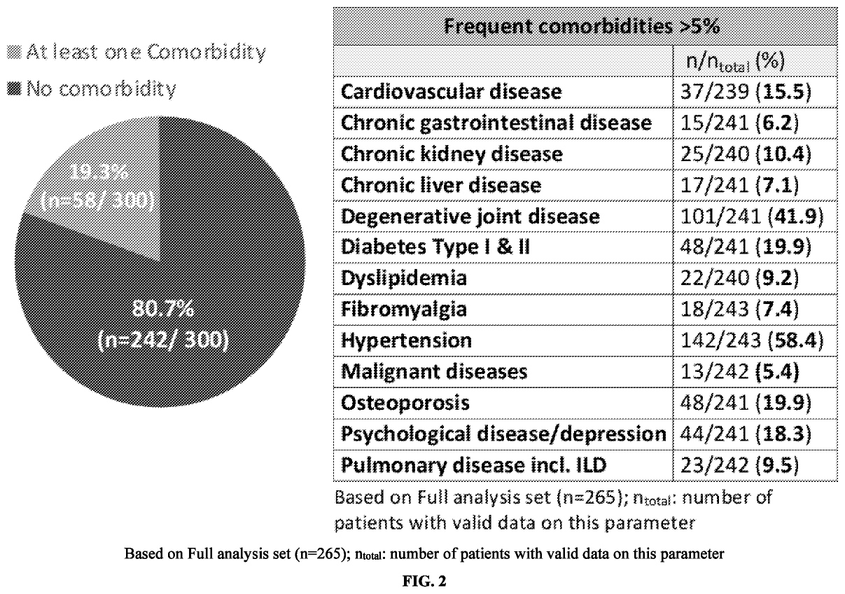

Data on comorbidities were available for 300 patients: comorbidities were present in 80.7% (n=242) of patients, frequent comorbidities were hypertension and cardiovascular disease, degenerative joint disease, osteoporosis, diabetes and depression. (See FIG. 2.)

At baseline, 32.5% (n=86) were biologic naive. Most common pretreatment with b/ts DMARDs included TNF-inhibitors (TNFi, 56.2%, n=149), non-TNFi biologics (29.1%, n=77) or JAK-inhibitors (JAKi, 17.4%, n=46) see Table 5. At baseline, 49% (n=149) received sarilumab as monotherapy and 29% (n=88) in combination with conventional synthetic DMARDs (csDMARDs) (comedication not specified for 22%, n=67). For 260 patients, autoantibody data were available: 47.3% (n=123) were RF⁺/ACPA⁺; 29.2% (n=76) were RF⁻/ACPA⁻ (see Table 4).

At treatment start, sarilumab was administered in 89.8% (n=273) at the standard dose of 200 mg q2w s.c.; 10.2% (n=31) were treated with 150 mg sarilumab q2w s.c. The autoinjector was used for 77.6% (n=236), while 22.4% (n=68) used the prefilled syringe as device.

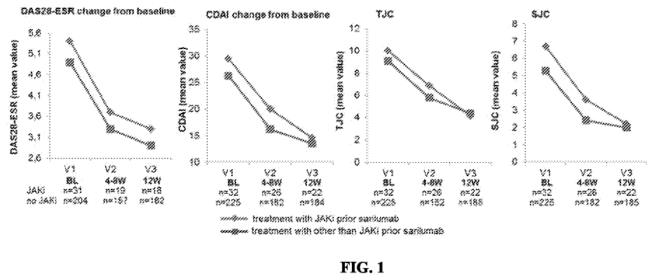

After 12 weeks of treatment with sarilumab, the mean DAS28-ESR decreased from 5.0±1.46 to 3.0±1.44 and CDAI from 26.7±13.79 to 13.6±11.4. DAS28-ESR remission/low disease activity was achieved in 42.8% (n=77/180 patients with valid data on this parameter)/59.4% (n=107/180) of subjects; 13.6% (n=28/206) and 49% (n=101/206) of subjects reached CDAI remission and low disease activity. Boolean remission was observed in 9.5% of subjects at week 12. HAQ-DI improved from 1.3 at baseline to 1.1 at week 12 (Table 4). The mean CDAI improvement was similar for autoantibody-positive (RF⁺ and/or ACPA⁺; CDAI −12.5 at week 12) compared to −negative subjects (CDAI −15.4 at week 12) (see Table 6). Subjects switching from JAKi to sarilumab (n=32), were more severely affected, had longer disease duration and received more prior treatments than subjects switched from another compound. Similar efficacy was observed among subjects that switched from JAKi to sarilumab vs subjects switched from other DMARDs; disease activity outcome measures including DAS28, CDAI, TJC, SJC and global assessments improved consistently (FIG. 1). 29 of the 32 JAKi treated subjects were switched from JAKi treatment because they were ineffectively treated with JAKi (lack of JAKi efficacy=12 and loss of JAKi efficacy=17). The JAKi treated subjects were treated with either baricitinib (n=13) or tofacitinib (n=19).

Safety was consistent with the anticipated profile of IL-6-R-inhibition and no new safety signals occurred. Adverse events and serious adverse events were described in 33.9% (118/348 patients) and 6.3% (22/348 patients) of subjects, respectively. Serious adverse drug reactions were documented in 3/348 patients (0.9%): injection site reactions (n=1), gastritis (n=1), and peritonitis (n=1). (see Table 7.)

TABLE 4
Incidence of remission and low disease activity in subjects
treated with sarilumab in routine care.

| parameter assessed | V1 (Baseline) | | V2 (4-8W) | | V3 (12W) | |
|---|---|---|---|---|---|---|
| DAS28-ESR | | | | | | |
| remission (<2.6); % [n/nₜₒₜₐₗ] | 4.7 | [11/235] | 32.4 | [57/176] | 42.8 | [77/180] |
| low disease activity (<3.2); | 11.5 | [27/235] | 49.4 | [87/176] | 59.4 | [107/180] |
| % [n/nₜₒₜₐₗ] | | | | | | |
| CDAI | | | | | | |
| remission (<2.8); % [n/nₜₒₜₐₗ] | 1.9 | [5/257] | 10.1 | [21/208] | 13.6 | [28/206] |
| low disease activity (<10); | 10.9 | [28/257] | 35.1 | [73/208] | 49.0 | [101/206] |
| % [n/nₜₒₜₐₗ] | | | | | | |
| Boolean Remission; % (n/nₜₒₜₐₗ) | 1.6 | [4/258] | 6.4 | [13/204] | 9.5 | [19/201] |
| TJC/SJC Mean (SD) | 9.2 (7.6)/5.5 (5.27) | | 5.9 (7.18)/2.6 (3.63) | | 4.3 (6.1)/2.0 (3.55) | |
| [nₜₒₜₐₗ] | [257] | | [208] | | [207] | |
| HAQ-DI Mean (SD) | 1.3 | (0.72) | 1.1 | (0.71) | 1.1 | (0.68) |

| | | | |
|---|---|---|---|
| [nₜₒₜₐₗ] | [256] | [200] | [195] |

Based on Full analysis set (n=265); Nₜₒₜₐₗ: number of patients with valid data on this parameter. DAS28-ESR, disease activity score in 28 joints involving erythrocyte sedimentation rate; CDAI, clinical disease activity index; TJC, tender joint counts; SJC, swollen joint counts; HAQ-DI, health assessment questionnaire disability index.

TABLE 5
Baseline data regarding patient characteristics,
prior therapy and autoantibodies.

| | | | | | Total | |
|---|---|---|---|---|---|---|
| | Monotherapy | | Sarilumab | | patient | |
| Characteristic | sarilumab | | +MTX | | population* | |
| Patient characteristics ^{#} | | | | | | |
| Sex female n/nₜₒₜₐₗ (%) | 120/149 | | 62/88 | | 232/304 | |
| | (80.5) | | (70.5) | | (76.3) | |
| Age Mean (SD), years | 59.4 | (11.6) | 58.2 | (10.3) | 58.6 | (11.2) |
| BMI Mean (SD) kg/m² | 27.6 | (6.1) | 28.3 | (6.0) | 28.4 | (6.3) |
| Time since diagnosis of RA Mean | 131.5 | (112.2) | 113.5 | (112.2) | 123.6 | (110.1) |
| (SD), months | | | | | | |
| Number of pre-specified prior RA | 5.5 | (2.3) | 4.6 | (2.3) | 5 | (2.2) |
| treatment mean (SD) ^{§} | | | | | | |
| Prior treatment before start of | nₜₒₜₐₗ = 130 | | nₜₒₜₐₗ = 80 | | nₜₒₜₐₗ = 265 | |
| sarilumab n(%) ^{§} | 127 | (97.7) | 77 | (96.3) | 254 | (95.8) |
| csDMARDs | 34 | (26.2) | 32 | (40.0) | 86 | (32.5) |
| Only csDMARD (no | | | | | | |
| bDMARD/tSDMARD) | | | | | | |
| TNFI | 81 | (62.3) | 43 | (53.8) | 149 | (56.2) |
| Other bDMARD (non TN Ft) | 40 | (30.8) | 15 | (18.8) | 77 | (29.1) |
| tsDMARD (JAKi) | 34 | (26.2) | 5 | (6.3) | 46 | (17.4) |
| Glucocorticoids | 115 | (88.5) | 66 | (82.5) | 219 | (82.6) |
| Combined RF/ACPA status ^{#} | nₜₒₜₐₗ = 130 | | nₜₒₜₐₗ = 72 | | nₜₒₜₐₗ = 260 | |
| RF+/CCP+ | 64 | (48.9) | 40 | (55.6) | 123 | (47.3) |
| RF+/CCP− | 12 | (9.2) | 9 | (12.5) | 34 | (13.1) |
| RF−/CCP+ | 17 | (13.0) | 5 | (6.9) | 27 | (10.4) |
| RF−/CCP− | 38 | (29.0) | 18 | (25.0) | 76 | (29.2) |

*only results for monotherapy, combination therapy and total patient population are displayed; results for patients with no specification mono/combination therapy are not shown.
^{#} based on baseline analysis set (n = 304),
^{§} based on Full analysis set (n = 265); ntotal: number of patients with valid data on this parameter.

Effectiveness by CDAI in dependence of autoantibody status

| | | Monotherapy | combination | total patient |
|---|---|---|---|---|
| | | mean (SD); | therapy mean | population |
| | Visit (week) | [nₜₒₜₐₗ] | (SD); [nₜₒₜₐₗ] | mean (SD); [nₜₒₜₐₗ] |
| RF⁺ or | V1 (start) | 25.6 (13.6); | 23.5 (13.7); | 25.5 (13.3); |
| ACPA⁺ | V3 (12 weeks) | [82] | [49] | [159] |
| | | 10.6 (9.3); | 13.1 (11.1); | 12.6 (12.0); |
| | | [63] | [40] | [121] |
| RF⁻ and | V1 (start) | 32 (13.4); | 27.9 (12.9); | 31.2 (13.2); |
| ACPA⁻ | V3 (12 weeks) | [29] | [15] | [63] |
| | | 17.1 (10.7); | 13.4 (7.5); | 17.1 (11.3); |
| | | [25] | [13] | [54] |

Based on full analysis set (n=265); *only results for monotherapy, combination therapy and total patient population are displayed; nₜₒₜₐₗ: number of patients with valid data.

TABLE 7
Safety Data

| | |
|---|---|
| n(%) | n₀ |

| | | | |
|---|---|---|---|
| Total | 348 | (100) | |
| Adverse event (AE) | 118 | (33.9) | 241 |
| Serious adverse event (SAE) | 22 | (6.3) | 28 |
| Adverse drug reaction (ADR)* | 53 | (15.2) | 91 |
| Serious adverse drug reaction | 3 | (0.9) | 4 |
| AE leading to study drug withdrawal | 43 | (12.4) | 67 |
| SAE leading to study drug withdrawal | 8 | (2.3) | 12 |

Based on safety set (n=348); *an AE was defined to be drug-related if the question “was AE related to product” (i.e. causal relationship) was answered “yes” with regard to sarilumab; drug-related by judgment of the treating rheumatologist.

Conclusions:

This data shows that treatment with sarilumab demonstrated rapid and clinically meaningful improvement in RA subjects that were ineffectively treated with JAKi. The safety profile was consistent with data reported from controlled clinical trials.

Patients switching from JAKi to sarilumab (n=32) had longer disease duration than patients switched from another compound (time since diagnosis of RA was 153.9 months for patients switched from JAKi vs. 123.3 months for patients switched from other DMARDs).

The most common reasons for switching from JAKi to sarilumab were loss of efficacy (n=17, 53.1%) and lack of efficacy (n=12, 37.5%)

Similar effectiveness was observed among patients that switched from JAKi to sarilumab vs. patients switched from other DMARDs. Disease activity outcome measures including DAS28, CDAI, TJC, SJC and global assessments improved consistently.

The compositions and methods of the present disclosure are not to be limited in scope by the specific embodiments describe herein. Indeed, various modifications of the present technology in addition to those described herein will become apparent to those skilled in the art from the foregoing description. Such modifications are intended to fall within the scope of the appended claims.

### Example 2

### Interim Analysis

The current interim analysis (IA) validates and expanding the insights into safety and effectiveness of sarilumab in patients with IR to last prior treatment JAKi (JAKi-IR). Additional subgroup analysis included patients who have received either TNF-inhibitor (TNFi) or non-TNFi bDMARD anytime in treatment history (bDMARD TH), and patients who have never received bDMARDs or targeted synthetic DMARDs (tsDMARDs) (b/tsDMARD naive).

439 patients were included who received at least one dose of sarilumab and for whom baseline documentation was accessible (baseline population, Table 8). For a portion of 388 patients, additional post-baseline efficacy data were available (full analysis population—JAKi-IR: n=65; TA: n=37; bDMARD TH: n=150; b/tsDMARD naive: n=136). All analyses are descriptive only.

At baseline, the mean (±SD) Clinical Disease Activity Index (CDAI) was 27.1±14.2 in JAKi-IR, 19.1±15.2 in TA, 23.0±12.7 in bDMARD TH, and 25.0±13.7 in b/ts DMARD naive (Table 8). Assessment of Health Assessment Questionnaire-Disability Index (HAQ-DI) at baseline revealed a mean (±SD) of 1.2±0.7 in JAKi-IR, 1.1±0.7 in TA, 1.2±0.7 in bDMARD TH and 1.2±0.8 in b/tsDMARD naive patients (Table 8).

Within the full analysis population 61.5% of JAKi-IR, 29.7% of TA, 50.0% of bDMARD TH and 52.9% of b/tsDMARD naive patients received glucocorticoids starting sarilumab therapy.

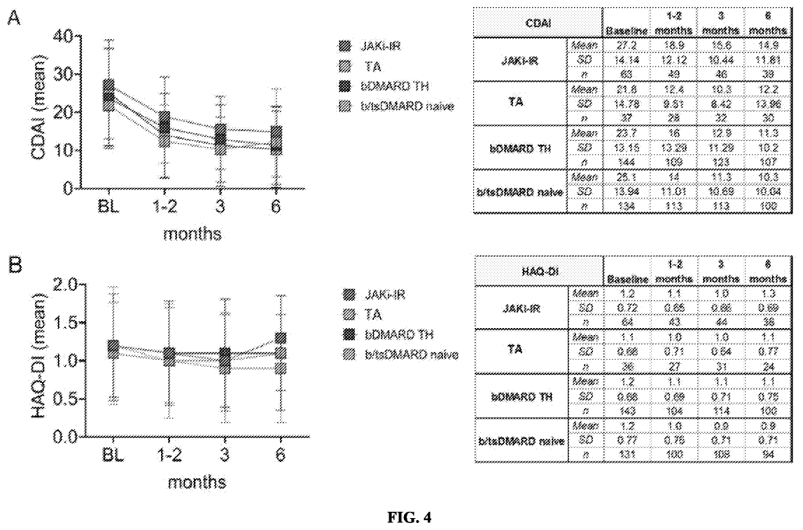

During 6 months of sarilumab treatment, CDAI improved in JAKi-IR (27.2±14.14 to 14.9±11.81) and TA (21.8±14.78 to 12.2±13.96) to the same extent as in the bDMARD TH cohort (23.7±13.15 to 11.3±10.2) and b/tsDMARD naive patients (25.1±13.94 to 10.3±10.04) (FIG. 4A). During the same time period HAQ-DI remained relatively stable (FIG. 4B).

Safety was consistent with the anticipated profile of sarilumab and without appearance of new signals. Adverse events (AEs)/serious AEs were reported in 60.0%/15.4%, 48.6%/16.2%, 56.7%/9.3% and 52.9%/11.0% of JAKi-IR, TA, bDMARD TH and b/tsDMARD naive patients, respectively.

Importantly, sarilumab administered in regular care demonstrated improvement of RA disease activity in patients with IR to JAKi as well as in bDMARD pretreated and b/tsDMARD naive patients. Without intending to be bound by scientific theory, the fact that no substantial change in physical function was observed might be due to lower baseline HAQ-DI scores in PROSARA compared to the phase III trials¹⁻³ and the relatively short observation time. Table 8: Baseline data regarding patient characteristics, disease activity and serostatus. Baseline population is shown. Therefore, baseline values might differ from baseline values in FIG. 4.

TABLE 8

| | | | | | | bDMARD | | b/tsDMARD | |
|---|---|---|---|---|---|---|---|---|---|
| | | JAKi-IR | | TA | | TH | | naive | |
| patient | patients [n] | 73 | | 47 | | 170 | | 149 | |
| characteristics | Sex female [n] (%) | 59 | (80.8) | 38 | (80.9) | 131 | (77.1) | 108 | (72.5) |
| | Age mean [years] (SD) | 58.7 | (9.7) | 58.8 | (13.6) | 58.5 | (11.6) | 59.3 | (10.7) |
| | BMI mean (SD) [kg/m²] | 29.4 | (6.0) | 26.8 | (5.2) | 28.5 | (6.3) | 28.1 | (6.4) |
| | Smoking history current | 23.6 | | 21.3 | | 25.0 | | 23.8 | |
| | [%] | | | | | | | | |
| | Time since diagnosis of RA | 12.2 | (8.7) | 12.6 | (7.7) | 12.9 | (10.4) | 6.5 | (7.4) |
| | mean [years] (SD) | | | | | | | | |
| disease | CRP [mg/l] (SD) | 8.7 | (10.6) | 11.7 | (46.5) | 12.2 | (20.5) | 18.7 | (39.2) |
| activity | ESR [mm/h] (SD) | 28.1 | (21.1) | 12.0 | (16.2) | 24 | (18.7) | 28.7 | (25.0) |
| | SIC mean (SD) | 6.5 | (5.7) | 3.5 | (4.6) | 4.3 | (4.6) | 4.9 | (5.2) |
| | TIC mean (SD) | 8.6 | (6.8) | 6.5 | (6.7) | 7.5 | (7.2) | 8.3 | (7.3) |
| | HAQ-DI mean (SD) | 1.2 | (0.7) | 1.1 | (0.7) | 1.2 | (0.7) | 1.2 | (0.8) |
| | DAS28-ESR mean (SD) | 5.1 | (1.4) | 3.4 | (1.8) | 4.6 | (1.4) | 4.9 | (1.5) |
| | CDAI mean (SD) | 27.1 | (14.2) | 19.1 | (15.2) | 23.0 | (12.7) | 25.0 | (13.7) |
| | Fatigue [VAS] (SD) | 56.7 | (23.3) | 47.1 | (30.0) | 52.8 | (27.9) | 54.4 | (29.0) |
| serostatus | RF+/CCP+ [n] (%) | 29 | (50.9) | 19 | (41.3) | 73 | (53.7) | 61 | (51.3) |
| | RF+/CCP− [n] (%) | 9 | (15.8) | 8 | (17.4) | 17 | (12.5) | 13 | (10.9) |
| | RF-/CCP+ [n] (%) | 4 | (7.0) | 6 | (13.0) | 18 | (13.2) | 6 | (5.0) |
| | RF−/CCP− [n] (%) | 15 | (26.3) | 13 | (28.3) | 28 | (20.6) | 39 | (32.8) |

### REFERENCES

1. Genovese M C, Fleischmann R, Kivitz A J et al. Sarilumab Plus Methotrexate in Patients With Active Rheumatoid Arthritis and Inadequate Response to Methotrexate: Results of a Phase III Study. Arthritis Rheumatol. 2015 June; 67(6):1424-37.
2. Fleischmann R, van Adelsberg J, Lin Y et al. Sarilumab and Nonbiologic Disease-Modifying Antirheumatic Drugs in Patients With Active Rheumatoid Arthritis and Inadequate Response or Intolerance to Tumor Necrosis Factor Inhibitors. Arthritis Rheumatol. 2017 February; 69(2): 277-290.
3. Burmester G R, Lin Y, Patel R et al. Efficacy and safety of sarilumab monotherapy versus adalimumab monotherapy for the treatment of patients with active rheumatoid arthritis (MONARCH): a randomised, double-blind, parallel-group phase III trial. Ann Rheum Dis. 2017 May; 76(5): 840-847.
4. Genovese M C, van der Heijde D, Lin Y et al. Long-term safety and efficacy of sarilumab plus methotrexate on disease activity, physical function and radiographic progression: 5 years of sarilumab plus methotrexate treatment. RMD Open. 2019 Aug. 1; 5(2):e000887.
5. European Medicines Agency. Kevzara (sarilumab)—summary of product characteristics, 2021. Available: haps://www.ema.europa.eu/en/documents/product-information/kevzara-epar-product-information_en.pdf [Accessed 18 Feb. 2021].
6. Feist E, Aries PM, Zinke S et al THU0165 PROSARA—A prospective, multicenter, non-interventional study to evaluate the safety and effectiveness of sarilumab for the treatment of active rheumatoid arthritis in regular care in Germany. Arthritis Rheumatol. 2020; 72 (suppl 10).

What is claimed is:

1. A method of treating rheumatoid arthritis in a subject that was previously ineffectively treated for rheumatoid arthritis with a JAK-inhibitor (JAKi), comprising administering an antibody that specifically binds to a human IL-6 receptor and comprises a heavy chain variable region (VH) and a light chain variable region (VL), wherein the VH comprises the three complementarity determining regions (CDRs) found within the amino acid sequence of SEQ ID NO:2 and wherein the VL comprises the three CDRs found within the amino acid sequence of SEQ ID NO:3.

2. The method of claim 1, wherein the subject is also administered a therapeutically effective amount of methotrexate.

3. The method of any one of claims 1-2, wherein the antibody is administered subcutaneously at a dose from about 150 mg to about 200 mg once every two weeks.

4. The method of claim 2, wherein the methotrexate is administered at a dose from 6 mg to 25 mg every week.

5. The method of any one of claims 1-4, wherein the antibody comprises three heavy chain complementarity determining region (HCDR) sequences comprising SEQ ID NOs: 4, 5, and 6 and three light chain complementarity determining region (LCDR) sequences comprising SEQ ID NOs: 7, 8 and 9.

6. The method of any one of claims 1-5, wherein the antibody is sarilumab.

7. The method of any one of claims 1-6, wherein the subject achieves a DAS28-ESR score below 3.1 after at least 12 weeks of administration of the antibody.

8. The method of any one of claims 1-7, wherein the subject achieves a clinical disease activity index (CDAI) score below 16 after at least 12 weeks of administration of the antibody.

9. The method of any one of claims 1-8, wherein the subject was previously ineffectively treated for rheumatoid arthritis with a JAKi selected from the group consisting of baricitinib, tofacitinib, and upadacitinib.

10. A method of treating rheumatoid arthritis in a subject that was previously ineffectively treated for rheumatoid arthritis with a JAKi, comprising administering a disease modifying antirheumatic drug (DMARD) selected from the group consisting of methotrexate, sulfasalazine, hydroxychloroquine and leflunomide, and an antibody that specifically binds to a human IL-6 receptor and comprises a heavy chain variable region (VH) and a light chain variable region (VL), wherein the VH comprises the three complementarity determining regions (CDRs) found within the amino acid sequence of SEQ ID NO:1 and wherein the VL comprises the three CDRs found within the amino acid sequence of SEQ ID NO:2.

11. The method of claim 10, wherein the antibody comprises three heavy chain complementarity determining region (HCDR) sequences comprising SEQ ID NOs: 4, 5, and 6 and 5, and three light chain complementarity determining region (LCDR) sequences comprising SEQ ID NOs: 7, 8 and 9.

12. The method of any one of claims 10-11, wherein the DMARD that is administered is methotrexate.

13. The method of any one of claims 10-12, wherein the antibody is administered subcutaneously at a dose from about 150 mg to about 200 mg once every two weeks.

14. The method of any one of claims 10-13, wherein the methotrexate is administered at a dose from 6 mg to 25 mg every week.

15. The method of any one of claims 10-14, wherein the antibody is sarilumab.

16. The method of any one of claims 10-15, wherein the subject achieves a DAS28-ESR score below 3.6 after at least 12 weeks of administration of the antibody and DMARD.

17. The method of any one of claims 10-16, wherein the subject achieves a clinical disease activity index (CDAI) score below 16 after at least 12 weeks of administration of the antibody and DMARD.

18. The method of any one of claims 10-17, wherein the subject was previously ineffectively treated for rheumatoid arthritis with a JAKi selected from the group consisting of baricitinib, tofacitinib, and upadacitinib.


SEQUENCE LISTING (11 sequences)

SEQ ID NO: 1 - protein, 358 aa, Homo sapiens
MVAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQD

SEQ ID NO: 2 - protein, 116 aa, Artificial Sequence
  source: /note=“Description of Artificial Sequence Synthetic polypeptide”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 3 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 4 - protein, 8 aa, Artificial Sequence
  source: /note=“Description of Artificial Sequence Synthetic peptide”
RFTFDDYA

SEQ ID NO: 5 - protein, 8 aa, Artificial Sequence
  source: /note=“Description of Artificial Sequence Synthetic peptide”
ISWNSGRI

SEQ ID NO: 6 - protein, 9 aa, Artificial Sequence
  source: /note=“Description of Artificial Sequence Synthetic peptide”
AKGRDSFDI

SEQ ID NO: 7 - protein, 6 aa, Artificial Sequence
  source: /note=“Description of Artificial Sequence Synthetic peptide”
QGISSW

SEQ ID NO: 8 - protein, 3 aa, Artificial Sequence
  source: /note=“Description of Artificial Sequence Synthetic peptide”
GAS

SEQ ID NO: 9 - protein, 9 aa, Artificial Sequence
  source: /note=“Description of Artificial Sequence Synthetic peptide”
QQANSFPYT

SEQ ID NO: 10 - protein, 446 aa, Artificial Sequence
  source: /note=“Description of Artificial Sequence Synthetic polypeptide”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  source: /note=“Description of Artificial Sequence Synthetic polypeptide”
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC